(19) United States
(12) Patent Application Publication
(10) Pub. No.: US 2007/0105179 A1
(43) Pub. Date: May 10, 2007

(54) Methods of detecting N-and O-linked oligosaccharides in glycoproteins

(75) Inventor: Michael Madson, Mountain View, CA (US)

Correspondence Address: David J. Brezner;Dorsey & Whitney LLP, Intellectual Property Department, 555 California Street, Suite 1000, San Francisco, CA, 94104-1513, US

(73) Assignee: Dionex Corporation

(21) Appl. No.: 11/270,258

(22) Filed: Nov. 9, 2005

(51) Int. Cl. C12Q 1/34; C12Q 1/37

(52) U.S. Cl. 435/18; 435/23

(57) ABSTRACT

Methods for removing N-linked and O-linked oligosaccharides from a glycoprotein including N-linked and O-linked oligosaccharides are provided. N-linked oligosaccharides are enzymatically cleaved from a glycoprotein to form cleaved-off N-linked oligosaccharides and residual glycoprotein. Residual glycoprotein is immobilized on a solid substrate. The cleaved-off N-linked oligosaccharides are separated from the residual glycoprotein. Subsequently, O-linked oligosaccharides are separated from the residual glycoprotein to form cleaved-off O-linked oligosaccharides and a residual protein. The cleaved-off O-linked oligosaccharides are separated from the residual protein. The N-linked and O-linked oligosaccharides are thus removed separately from the glycoprotein, and can be detected separately.

### FIELD

Methods, systems, and kits for removing N-linked and O-linked oligosaccharides from glycoproteins are provided.

### BACKGROUND

Post-translational modifications of proteins affect protein structure and activity. In particular, proteins are often post-translationally modified by attaching oligosaccharides chains. Oligosaccharides that are covalently attached to proteins generally fit into one of two broad categories: N-linked oligosaccharides and O-linked oligosaccharides. N-linked oligosaccharides are oligosaccharides that are covalently attached to an amide nitrogen on an asparagines residue. O-linked oligosaccharides are oligosaccharides that are covalently attached to serine or threonine residues on a glycoprotein.

Two standard methods for the detecting of N- and O-linked oligosaccharides include hydrazine hydrolysis and concentrated ammonia/ammonium carbonate hydrolysis. In Biochemistry (1993) 32 679-693, Patel et al. use hydrazine to generate both N- and O-linked oligosaccharides from glycoproteins. Huang et al., Anal. Chem. 73 (24) (2001) use a combination of concentrated ammonia and ammonium carbonate to release both N- and O-linked oligosaccharides in the same mixture. Packer and Karlsson in US Patent Publication US/2004/0039192 A1 and Packer and Karlsson in International Patent Application WO 02/06295 A1 disclose separating O-linked oligosaccharides from glycoprotein via alkaline beta elimination with an apparatus that uses bound glycoprotein and a flow of alkali, neutralization with cation exchange resin and concentration/collection with carbon column. Hounsell, Stoll, Feizi and Lawson in International Patent Application /Publication WO 90/04596 May 3, 1990 use reductive beta elimination and concentration of O-linked oligosaccharides with the use of a silica based phenyl boronic acid column coupled to periodate treatment of the released oligosaccharide alditol at the C4-C5 position of the substituted alditol and coupling to a lipid to assist detection. In Anal. Biochem. (2003) 131-134, Rohrer and Miller use an endo α N-acetyl galactosamidase to release galactose β (1, 3) N-acetyl Galactosamine, the most common O-linked oligosaccharide, from the 0-glycopeptide. High-pH anion-exchange chromatography (HPAEC) of N-linked oligosaccharides has been documented, HPAEC separation of O linked oligosaccharides has not been performed.

None of these methods provide for separate cleavage of N-linked and O-linked oligosaccharides. Further, these methods often result in the degradation of oligosaccharides. There is a need for simple methods, systems, and kits for detecting, analyzing, and/or identifying N- and O-linked oligosaccharides from a single sample of glycoprotein that separates the N-linked and O-linked oligosaccharides non-destructively prior to detection. The present application addresses these and other needs.

### SUMMARY

The present invention provides methods of detecting N-linked and O-linked oligosaccharides from a glycoprotein. In one aspect of the invention, one or more N-linked oligosaccharides are enzymatically cleaved from the glycoprotein leaving the O-linked oligosaccharides attached to the residual glycoprotein. The residual glycoprotein is immobilized on a solid substrate, and the cleaved-off N-linked oligosaccharides are separated from the residual glycoprotein. Optionally, the residual glycoprotein can be eluted from the solid substrate and collected for analysis.

The O-linked oligosaccharides are then cleaved from the separated residual glycoprotein to form cleaved-off O-linked oligosaccharides and a residual protein. In certain embodiments, the O-linked oligosaccharide is cleaved by reductive beta-elimination within one or more specific pH ranges. The cleaved-off O-linked oligosaccharides are separated from the residual protein (e.g. on an ion exchange column), and the separated N-linked and O-linked oligosaccharides are detected.

In certain embodiments, the methods are further directed to reducing the cleaved-off N-linked oligosaccharides and/or O-linked oligosaccharides to form cleaved-off N-linked and O-linked oligosaccharide alditols, respectively. For example, the cleaved-off N-linked or O-linked oligosaccharides may be contacted with a borohydride, such as NaBH₄. Further, cleaved-off N-linked or O-linked oligosaccharides, or their respective alditols, can be optimized for detection by various detection methods.

### BRIEF DESCRIPTION OF THE DRAWINGS

FIG. 1 depicts a flow diagram of one embodiment of a method of removing N-linked and O-linked oligosaccharides from a glycoprotein;

FIG. 2 depicts a flow diagram showing an embodiment of removing N-linked and O-linked oligosaccharides from a glycoprotein;

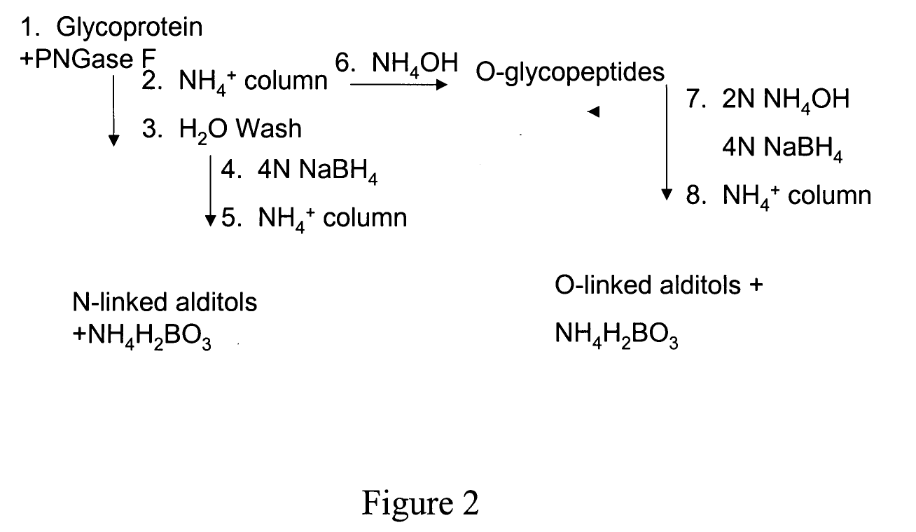

FIG. 3 depicts the hypothetical mechanism of beta-elimination and reduction of an O-linked oligosaccharide;

FIG. 4 depicts negative ion electrospray ionization (ESI) mass spectrum of N-linked Oligosaccharide Alditols cleaved and separated from fetuin;

FIG. 5 depicts a negative ion ESI-mass spectrum of O-linked Oligosaccharide Alditols cleaved and separated from κ Casein;

FIG. 6 depicts a negative ion ESI-mass spectrum of O-linked oligosaccharide alditols cleaved and separated from K Casein, and further isolated in a first fraction by ion exchange chromatography; and

FIG. 7 depicts a negative ion ESI-mass spectrum of O-linked oligosaccharide alditols cleaved and separated from K Casein, and further isolated in a second fraction by ion exchange chromatography.

### DETAILED DESCRIPTION

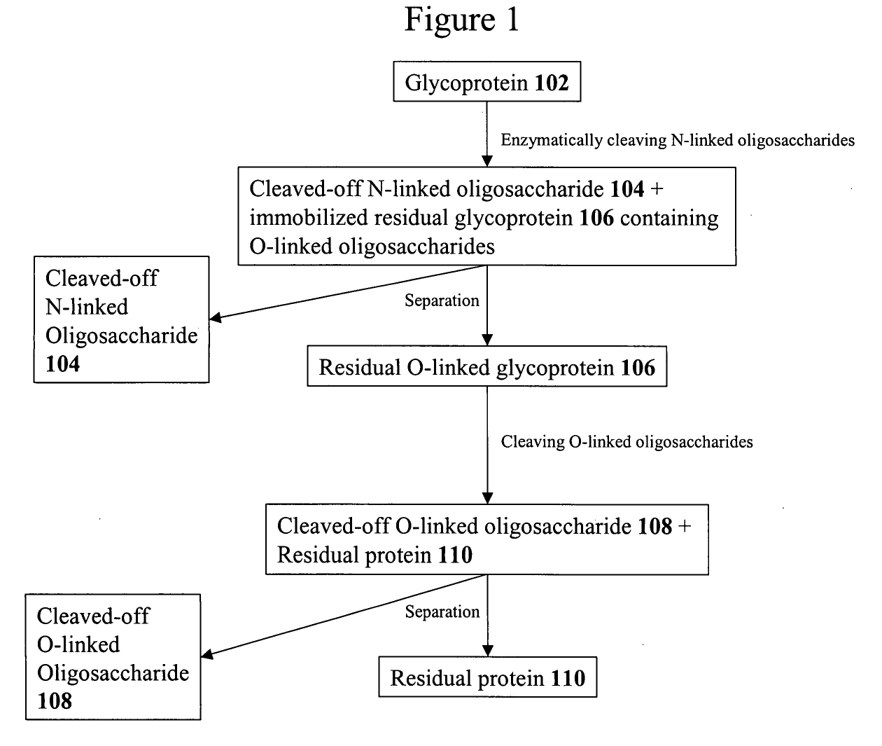

The present invention provides methods for removing N-linked and O-linked oligosaccharides from a glycoprotein including N-linked and O-linked oligosaccharides. With reference to flow diagram 100 of FIG. 1, N-linked oligosaccharides are enzymatically cleaved from a glycoprotein 102 to form cleaved-off N-linked oligosaccharides 104 and residual glycoprotein 106. Residual glycoprotein 106 is immobilized on a solid substrate (e.g. an ion exchange column). The cleaved-off N-linked oligosaccharides 104 are then separated from the residual glycoprotein 106, preferably by washing, and preferably collected. Subsequently, O-linked oligosaccharides are cleaved from the residual glycoprotein 106 to form cleaved-off O-linked oligosaccharides 108 and a residual protein 110. The cleaved-off O-linked oligosaccharides 108 are then separated from the residual protein 110. The N-linked oligosaccharides 104 and O-linked oligosaccharides 108 are thus removed separately from the glycoprotein 102, and can be detected separately. As used herein, the terms “detecting” or “detection” of the separated O-linked and/or N-linked oligosaccharides refers to detection of these cleaved-off compounds, derivatives of these compounds, or fragments of these compounds.

Previous methods either cleave N-linked and O-linked glycoproteins simultaneously or degrade N-linked and/or O-linked oligosaccharides. By contrast, the methods disclosed herein provide for separating cleaved-off N-linked oligosaccharides from cleaved-off O-linked oligosaccharides, such that the cleaved-off N-linked and O-linked oligosaccharides can be separately detected. Further, the cleaved-off N-linked and O-linked glycoproteins are not degraded prior to detection.

By “glycoprotein” is meant a protein-oligosaccharide compound where the protein and oligosaccharide portion are covalently linked. As used herein, the protein portion of the glycoprotein includes at least two covalently attached amino acids. The protein portion can be made up of naturally occurring amino acids or non-naturally occurring amino acids. By “oligosaccharide” is meant at least two monosaccharide sugars covalently linked together. Oligosaccharides in glycoproteins are generally N-linked oligosaccharides and O-linked oligosaccharides. By “N-linked oligosaccharide” is meant an oligosaccharide that is covalently attached to an asparagine residue of the glycoprotein. By “O-linked oligosaccharide” is meant an oligosaccharide covalently attached to a serine or threonine residue of a glycoprotein. Exemplary glycoproteins include κ casein, thyroglobulin, the secretions of mucous membranes (i.e. mucins), erythropoietin (EPO), and fetuin.

The methods herein are preferably performed in aqueous liquid solutions. Further, the methods described herein preferably maintain the stability of the N-linked and O-linked oligosaccharides such that they can be readily detected.

A. Enzymatically Cleaving the N-Linked Oligosaccharides

N-linked oligosaccharides are enzymatically cleaved from the glycoprotein to form cleaved-off N-linked oligosaccharides and residual glycoprotein. By the phrase “cleaved-off N-linked oligosaccharides” is meant the oligosaccharides cleaved from an amide nitrogen of an asparagine residue of the glycoprotein. By the term “residual glycoprotein” is meant the portion of the glycoprotein which includes the protein portion and O-linked oligosaccharide portion, but which does not include N-linked oligosaccharide. The term residual protein includes within its scope glycoprotein residues having some uncleaved N-linked oligosaccharides.

The N-linked oligosaccharides are enzymatically cleaved from the glycoprotein is accomplished by contacting the glycoprotein with an enzyme capable of cleaving N-linked oligosaccharides but not the O-linked oligosaccharides from a glycoprotein. Preferably, this process is performed in an aqueous medium. The enzyme can be any enzyme capable of cleaving N-linked oligosaccharides but not O-linked oligosaccharides. In a preferred embodiment, the enzymatically cleaving step includes contacting the glycoprotein with an enzyme selected from the group consisting of PNGase F and PNGase A. Both PNGase F (New England Biolabs, Beverly Mass.) and PNGase A (New England Biolabs, Beverly Mass.) cleave the asparagine-oligosaccharide linkage in glycoproteins. Cleavage occurs at the amide bond between the nitrogen group on asparagine and the oligosaccharide. Suitably, the conditions of cleavage with these enzymes are described in the product literature of these enzyme manufacturers. For example, cleavage can occur at 37° C. over a 12 hour period.

B. Immobilizing the Residual Glycoprotein on a Solid Substrate

The residual glycoprotein is then disposed on a solid substrate. By “solid substrate” is meant a solid in any form that is capable of binding residual protein. Exemplary solid substrates can include any strong cation exchange resin. Alternative exemplary solid substrates include hydrophobic portions, and can bind to hydrophobic portions of the glycoproteins. The substrate can be in any conventional form such as a packed bed, a membrane, or a macroporous monolith.

In certain preferred embodiments, positively charged portions of the O-linked glycoprotein residue bind to a negatively charged solid substrate, e.g. cation exchange resin in a bed. The electrostatic attraction between the positively charged glycoprotein residue and negatively charged solid substrate results in the retention of the glycoprotein residue. The cleaved-off N-linked oligosaccharides have a neutral or positive charge, and are separated from the substrate. The cleaved-off N-linked oligosaccharides can be separated from the glycoprotein residue based on their respective ionic properties.

Alternatively, the solid substrate includes hydrophobic portions. Hydrophobic glycoprotein residues can bind to the solid substrate by van der Waals interactions or the like.

In certain embodiments, the solid substrate (e.g. resin bed) can be disposed in a housing, such as a housing used in a column or cartridge. Exemplary columns and cartridges include the ON GUARD II H cartridge, cation form (Dionex, Calif.). In a preferred embodiment, the solid substrate is the stationary phase of said cation exchange column. Cations forms include hydrogen, ammonium, sodium, and potassium forms. In a particularly preferred embodiment, the cation exchange column is an ammonium exchange column.

The order of cleaving the N-linked oligosaccharides and immobilizing the glycoprotein to the solid substrate can vary. In a preferred embodiment, the residual protein is immobilized to the solid substrate after cleaving the N-linked oligosaccharides from the glycoprotein in aqueous solution. Alternatively, the glycoprotein can be immobilized to the solid substrate before or simultaneously to cleaving the N-linked oligosaccharides from the glycoprotein.

C. Separating the Cleaved-off N-Linked Oligosaccharides from the Residual Glycoprotein

The cleaved-off N-linked oligosaccharides are then separated from the residual glycoprotein by any separation technique or method known in the art. In embodiments where the solid substrate is the stationary phase of a column or cartridge and is bound by electrostatic forces, the cleaved-off N-linked oligosaccharides can be separated by flowing an aqueous liquid stream past the solid substrate. The aqueous liquid stream carries the cleaved-off N-linked oligosaccharides, leaving the residual glycoprotein immobilized on the solid substrate.

The cleaved-off N-linked oligosaccharide can be further processed for ease of detection after it is separated from the residual glycoprotein. For example, the cleaved off N-linked oligosaccharides can be reduced to form cleaved-off N-linked oligosaccharide alditols. Reduction of the cleaved-off N-linked oligosaccharide can be accomplished by any method known in the art. In one embodiment, the cleaved-off N-linked oligosaccharide is contacted with a reducing agent that reduces, but does not otherwise degrade, the cleaved-off N-linked oligosaccharide. Examples of such reducing agents include borohydrides. In one such embodiment, the borohydride is NaBH₄. In this embodiment, the reduced cleaved-off N-linked oligosaccharide alditol is in solution with NaH₂BO₃.

After the cleaved-off N-linked oligosaccharide has been removed from the residual glycoprotein, the residual glycoprotein can then be removed from the solid substrate. The removal of the residual glycoprotein can be accomplished by any method known in the art. For example, the residual glycoprotein can be eluted by providing to the solid substrate, a basic eluent, e.g. at a pH of about 11 to 12, such as ammonium hydroxide, e.g. at a 2N concentration.

D. Cleaving O-Linked Oligosaccharides from the Residual Glycoprotein to Form Cleaved-off O-Linked Oligosaccharides and a Residual Protein

The O-linked oligosaccharides are cleaved from the residual glycoprotein to form cleaved off O-linked oligosaccharides and residual protein. By the phrase “cleaved-off O-linked oligosaccharide” is meant an oligosaccharide formerly covalently bonded to a serine or threonine residue, but that is no longer covalently bonded to the serine or threonine residue. By the phrase “residual protein” herein is meant the portion of the glycoprotein remaining after cleaving N-linked and O-linked oligosaccharides. The phrase “residual protein” includes within its scope proteins having some uncleaved O-linked oligosaccharides. At least a portion of the O-linked oligosaccharides on the glycoprotein have been cleaved to form the residual protein.

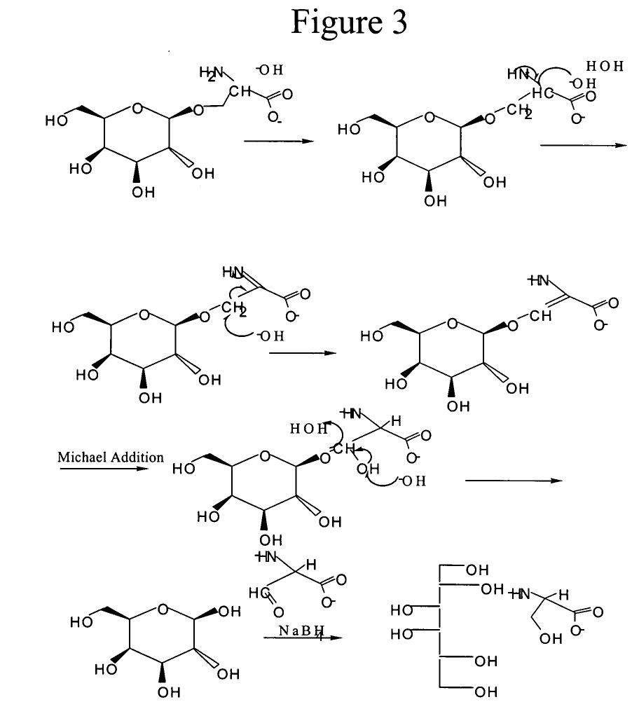

The O-linked oligosaccharide can be removed from the residual glycoprotein by any enzymatic or chemical method known in the art. In certain embodiments, cleaving the O-linked oligosaccharides from the residual glycoprotein can be accomplished by beta-elimination of the O-linked oligosaccharide from the residual glycoprotein. Without wishing to be limited to a particular mechanism or theory, FIG. 3 depicts a putative reaction mechanism of beta-elimination of an oligosaccharide from a serine amino acid residue.

Cleaving the O-linked oligosaccharides from the residual glycoprotein by beta-elimination can be accomplished using any basic solution known in the art. In certain embodiments, cleaving the O-linked oligosaccharide can be accomplished by contacting the residual glycoprotein containing the O-linked oligosaccharide with an hydroxide solution. Any hydroxide solution known in the art may be used. For example, cleaving the O-linked oligosaccharides is performed in a base such as NaOH, KOH, or NH₄OH. In certain embodiments, the O-linked oligosaccharide may be performed for a period of hours, e.g. 16 hours or more, at a specific pH. In certain embodiments, cleavage of O-linked oligosaccharides from residual glycoprotein can occur at a pH of between 11 and 12, e.g. at 11.4, for a specific period of time (e.g. 16 hours). In a preferred embodiment, the cleaving the O-linked oligosaccharides from the residual glycoprotein is performed in an NH₄OH solution, such as a 2N NH₄OH solution.

Cleaving O-linked oligosaccharides from residual glycoprotein by beta-elimination can be performed without degrading the cleaved-off oligosaccharide by controlling the pH of the cleaving step. The pH of the beta-elimination reaction can be held within a range of pH values such that the O-linked oligosaccharides are cleaved from the residual glycoprotein, but do not degrade the O-linked oligosaccharides. In certain embodiments, the step of cleaving the O-linked oligosaccharides can be performed at a pH that is greater than or equal to about 8.5, 8.6, 8.7, 8.8, 8.9, 9.0, 9.1, 9.2, 9.3, 9.4, 9.5, 9.6, 9.7, 9.8, 9.9, 10.0, 10.1, 10.2, 10.3, 10.4, 10.5, 10.6, 10.7, 10.8, 10.9, 11.0, 11.1, 11.2, 11.3, 11.4, 11.5, 11.6, or 11.7. In further embodiments, the step of cleaving the O-linked oligosaccharides can be performed at a pH that is less than or equal to about 11.8, 11.7, 11.6, 11.5, 11.4, 11.3, 11.2, 11.1, 11.0, 10.9, 10.8, 10.9, 10.8, 10.7, 10.6, 10.5, 10.4, 10.3, 10.2, 10.1, 10.0, 9.9, 9.8, 9.7, 9.6, 9.5, 9.4, or 9.3, 9.2 or 9.1 . The pH of the solution can be adjusted using any basic or acidic compounds known in the art.

In certain embodiments, the step of cleaving the O-linked oligosaccharides from the residual glycoprotein can be performed within a single pH range. For example, in certain embodiments, the step of cleaving the O-linked oligosaccharides from the residual glycoprotein can performed at a pH between about 9.1 and 9.8. Alternatively, the step of cleaving the O-linked oligosaccharides from the residual glycoprotein can performed within one of multiple pH ranges. For example, the step of cleaving the O-linked oligosaccharides from the residual glycoprotein can performed at a pH between about 9.1 and 9.8 or about 10.2 and 10.8. Optimal pH ranges can depend on a number of factors, including the glycoprotein and the chemical structure of the O-linked oligosaccharide. The optimal pH can be readily ascertained by those skilled in the art.

E. Separating the Cleaved-off O-Linked Oligosaccharides from the Residual Protein.

After the O-linked oligosaccharides are cleaved from the residual glycoprotein, the cleaved-off O-linked oligosaccharides are separated from the residual protein. Separating the cleaved-off O-linked oligosaccharides from the residual protein can be accomplished by any method known in the art.

In one embodiment, separating the O-linked oligosaccharides is performed by immobilizing residual glycoprotein on a solid substrate. For example, he solid substrate can be the stationary phase of a column or cartridge. Separating the cleaved-off O-linked oligosaccharides from the residual protein can be performed by flowing a liquid stream past the solid substrate after cleaving the O-linked oligosaccharides from the residual protein. The O-linked oligosaccharides are then carried away from the residual protein, leaving the residual protein immobilized on the solid substrate.

In a preferred embodiment, the solid substrate is the stationary phase of the cation exchange column. The cleaved-off O-linked oligosaccharide and the residual protein are separated based because of their different electrostatic charges. The positively charged residual protein is immobilized on the negatively charged stationary phase, and the neutral or negatively charged cleaved-off O-linked oligosaccharides are removed from the column with water or a basic solution. In preferred embodiments, the cation exchange column is an ammonium exchange column.

In another embodiment, the solid substrate can have hydrophobic moieties and can bind the protein, e.g. by van der Waal's interactions. The cleaved-off O-linked oligosaccharides can then be separated base on hydrostatic interactions of the protein residue with the column. Combinations of electrostatic and hydrophobic binding may be employed.

The order of cleaving the O-linked oligosaccharides and immobilizing the residual glycoprotein to the solid substrate can vary. In certain embodiments, O-linked oligosaccharides can be removed from the residual glycoprotein, and the residual protein can be immobilized on the solid substrate. Alternatively, the glycoprotein residue that includes the O-linked oligosaccharides can be immobilized on the solid substrate, and the O-linked oligosaccharides can then be cleaved from the glycoprotein. In still other embodiments, the glycoprotein can remain immobilized on the solid substrate after separation from the N-linked oligosaccharides. The O-linked oligosaccharides can then cleaved from the still-immobilized glycoprotein, and the O-linked oligosaccharides can be separated from the immobilized protein residue.

Like the cleaved-off N-linked oligosaccharides, the cleaved-off O-linked oligosaccharides can be further processed after separation from the residual glycoprotein. For example, the cleaved off O-linked oligosaccharides can be reduced (e.g. in situ) to form cleaved-off O-linked oligosaccharide alditols.

Like reduction of the cleaved-off N-linked oligosaccharides, reduction of the cleaved-off O-linked oligosaccharide can be accomplished by any method known in the art. In one embodiment, the cleaved-off O-linked oligosaccharides are contacted with a reducing agent that reduces, but does not otherwise degrade, the cleaved-off O-linked oligosaccharide. Examples of such reducing agents include borohydrides such as NaBH₄. The reduced cleaved-off O-linked oligosaccharide alditol results in formation of NaH₂BO₃.

F. Detecting N-Linked and O-Linked Oligosaccharides

The N-linked and O-linked oligosaccharides from the glycoproteins or derivatives thereof can be detected by any method known in the art. The N-linked and O-linked oligosaccharides are separated as described herein. For example, the N-linked and O-linked oligosaccharides can be detected by high performance liquid chromatography (HPLC) and/or mass spectrometry. Exemplary HPLC separation methods include use of ion exchange columns (e.g. a Carbopac PA20 ion exchange column). Exemplary mass spectrometry methods include MALDI or ESI mass spectrometry.

The N-linked and O-linked oligosaccharides can be processed to optimize detection by individual detection techniques. The additional processing of the oligosaccharides depends on the detection method used. For example, in certain embodiments, the cleaving, immobilizing, and separating steps involve the use of sodium, potassium, and other non-volatile cations. Such non-volatile cations, however, can interfere with ionization and detection of the oligosaccharides in matrix assisted laser desorption/ionization (MALDI) and electrospray ionization (ESI) mass spectrometry. To optimize detection by mass spectrometry, sodium and potassium ions are replaced with more readily volatile NH₄⁺ ions. The NH₄⁺ ions can be substituted in any format, including chromatography (e.g. ion exchange chromatography), repeated washing with an ammonium solution, and other methods.

The N-linked oligosaccharides can be detected batchwise, without further separation. Alternatively, the N-linked oligosaccharides can be separated further as is known in the art. Similarly, the O-linked oligosaccharides can be detected batchwise without further separation, or can be separated further prior to detection.

Methods of separating N-linked oligosaccharides from each other and O-linked oligosaccharides from each other are well known in the art. For example, different cleaved-off N-linked oligosaccharides can be separated by chromoatography. Similarly, different cleaved-off O-linked oligosaccharides can be separated by chromatography. In certain embodiments, separation of oligosaccharide alditols can be accomplished with any column know in the art. In a preferred embodiment, the column is an ion exchange column (e.g. Carbo Pac PA 200 column or a Carbo Pac MA1 column, Dionex, Sunnyvale, Calif.).

In certain embodiments, the monosaccharide composition of at least one of the cleaved-off N-linked oligosaccharides, O-linked oligosaccharides, or alditols thereof are detected. For example, the cleaved-off N-linked, O-linked oligosaccharides, or alditols thereof can be detected directly. The cleaved-off N-linked, O-linked oligosaccharides, or alditols thereof can also be degraded by exo or endo-glycosidase, to either to monosaccharides or larger subunits, which can then be detected.

In a preferred embodiment, the total number of specific monosaccharide units can be determined by graded hydrolysis to separate monosaccharides. In other embodiments, the sequence of oligosaccharides can be determined. For example, the oligosaccharides can be cleaved by endo- or exo-glycosidases at specific positions, and the products can be detected. The specific monosaccharide at each successive position in a sequence of monosaccharides can be determined.

In a further embodiment, the composition and/or sequence of oligosaccharides can be determined directly by mass spectrometry. The mass of cleaved-off N-linked or O-linked oligosaccharide, alditols thereof, and/or degradation products can be determined by mass spectrometry. The gas-phase decay of certain peaks can be further analyzed to determine sequence information by tandem mass spectrometry, as is known in the art.

The present invention is also directed to kits that include one, two, three, or more components used to practice the methods disclosed herein. Specifically, the kits can include one or more enzymes capable of cleaving N-linked oligosaccharides from a glycoprotein containing N-linked oligosaccharides, solid substrates, and/or columns for separating cleaved-off N-linked oligosaccharides and cleaved off O-linked oligosaccharides from glycoprotein and protein residues. Kits can further include columns or cartridges used to purify or process N-linked and O-linked oligosaccharides, and columns or cartridges used prepare oligosaccharides for detection. Kits can further include instructions for detecting N-linked and O-linked oligosaccharides according to the methods disclosed herein. The kit components can be provided in any number and combination.

### EXAMPLES

The following examples serve to more fully describe the manner of using the above-described invention, as well as to set forth the best modes contemplated for carrying out various aspects of the invention. It is understood that these examples in no way serve to limit the true scope of this invention, but rather are presented for illustrative purposes. All references cited herein are incorporated by reference.

### Example 1

A 1 mg sample of κ Casein was combined with 1 microliter (484 U) PNGase F (New England Biolabs, Beverly Mass.) to cleave N-linked oligosaccharides from the Kc Casein. The reaction was allowed to proceed overnight at 37° C.

The mixture was then placed on an ON GUARD II H, ammonium form ion exchange column. Cleaved-off N-linked oligosaccharides were eluted by pumping 2 ml water over the column, and the cleaved-off N-linked oligosaccharides were recovered. The cleaved-off N-linked oligosaccharides were reduced in 4N NaBH₄ to form cleaved-off N-linked oligosaccharide alditols and NaH₂BO₃. The cleaved-off N-linked oligosaccharide alditols and NaH₂BO₃ were passed over a second ammonium exchange column to remove the Na from the NaH₂BO₃ to form NH₄H₂BO₃.

The glycoprotein residue still containing O-linked oligosaccharides was then eluted from the first ammonium exchange column with 1.7 ml, 2N NH₄OH. 0.2 mL 2N NH₄OH was added to the glycoprotein residue solution containing 0.003 ml 4N NaB₄ to cleave the O-linked oligosaccharides, and the mixture was allowed to incubate for 16 hours to form cleaved-off O-linked oligosaccharide alditols and NaH₂BO₃. The cleaved-off O-linked oligosaccharide alditols and NaH₂BO₃ were passed over a second ammonium exchange column to remove the Na from the NaH₂BO₃ to form NH₄H₂BO₃.

The eluted N-linked glycoproteins and O-linked glycoproteins were then detected by mass spectrometry.

### Example 2

A bovine fetuin sample was obtained, and the N-linked oligosaccharides were obtained in a manner analogous to the method of Example 1.

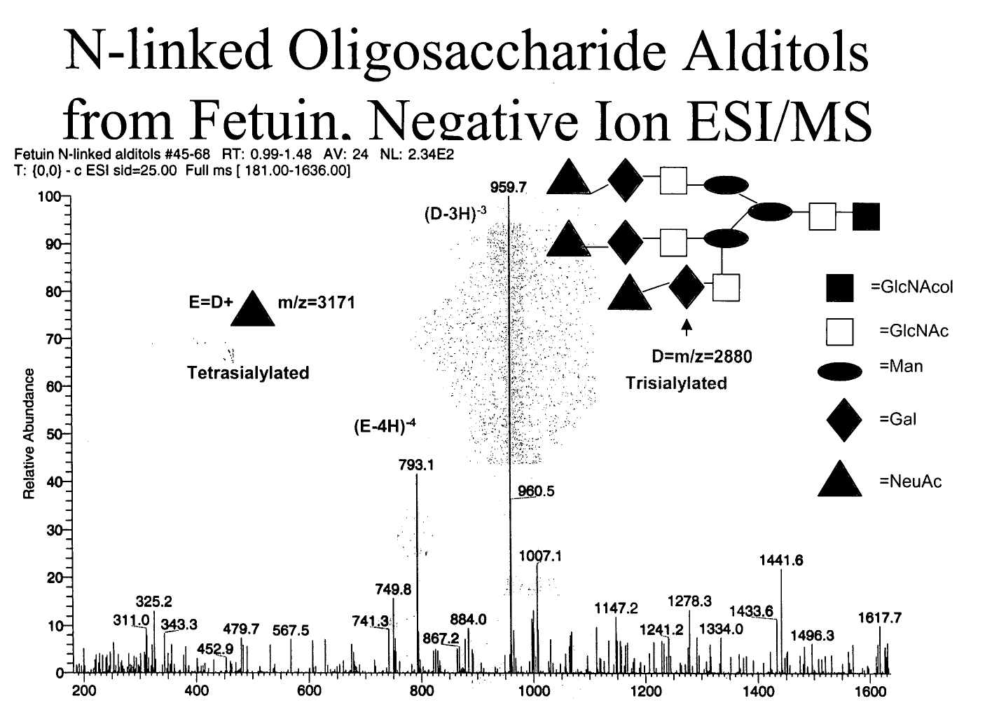

The cleaved-off N-linked oligosaccharides were then analyzed by ESI mass spectrometry. The mass spectrum is depicted in FIG. 4. Trisilated and tetrasilated N-linked oligosaccharides were detected.

### Example 3

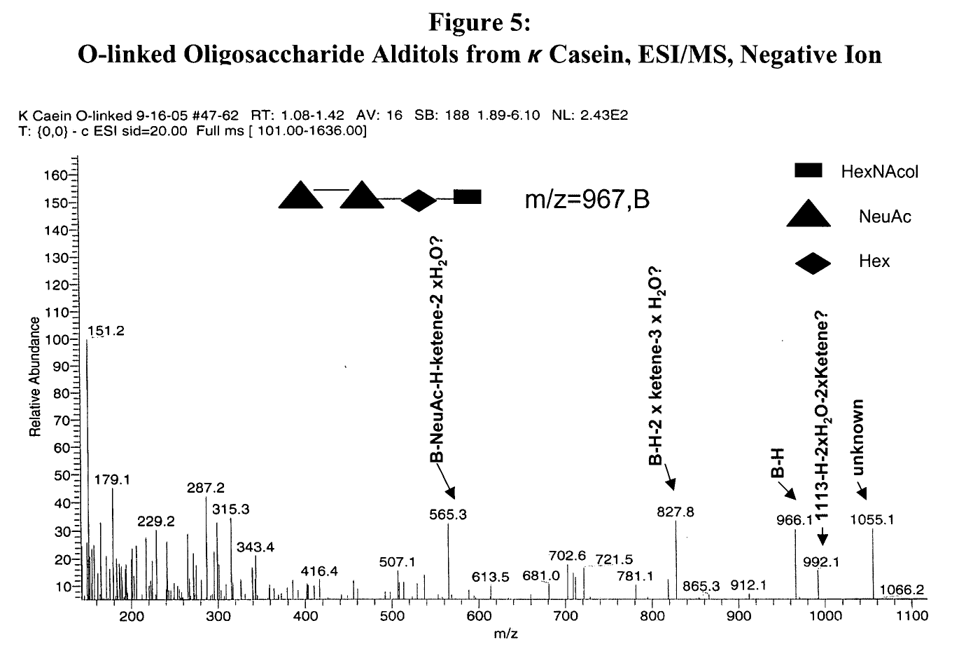

Cleaved-off O-linked Oligosaccharide alditols from κ Casein were obtained as described in Example 1. The cleaved-off O-linked alditols were then detected by ESI-mass spectrometry in negative ion mode. The mass spectrum is depicted in FIG. 5.

FIG. 5 shows a mass spectrum with mass peaks corresponding to the full length NeuAc-NeuAc-Glc-HexNAc (SEQ ID NO:1) cleaved-off O-linked oligosaccharide. The composition and sequence of the oligosaccharide can thus be deduced from the detected masses.

### Example 4

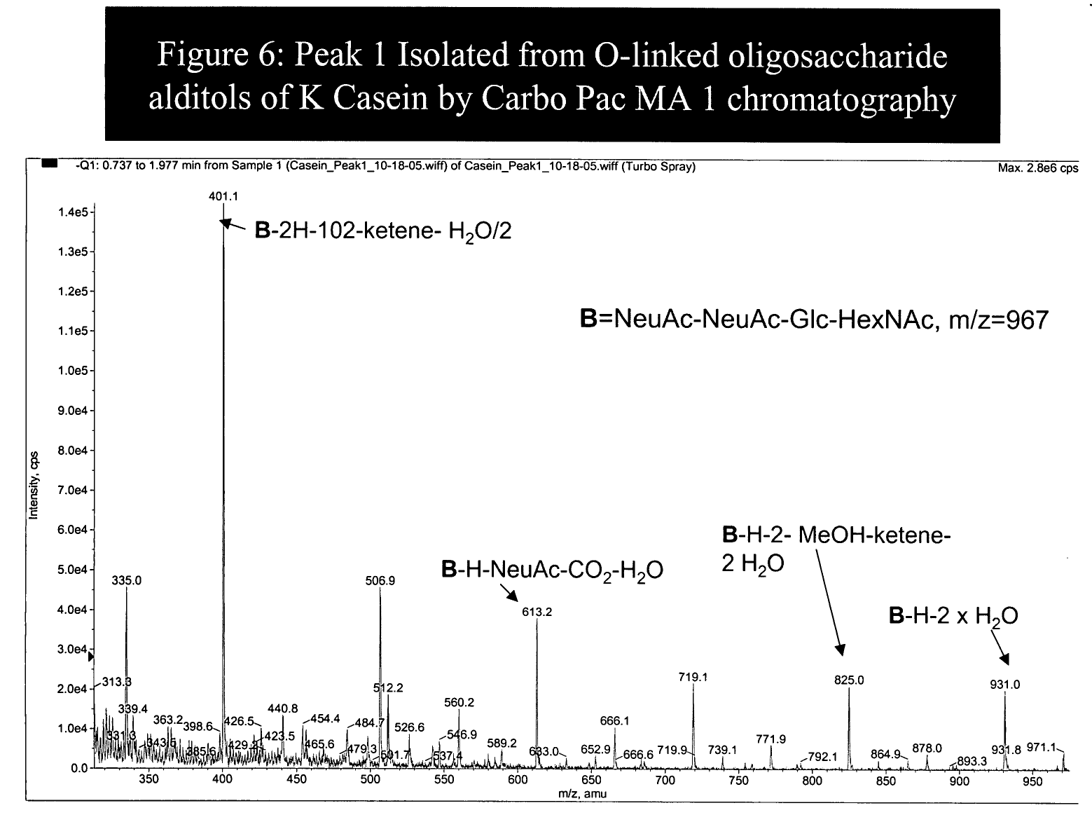

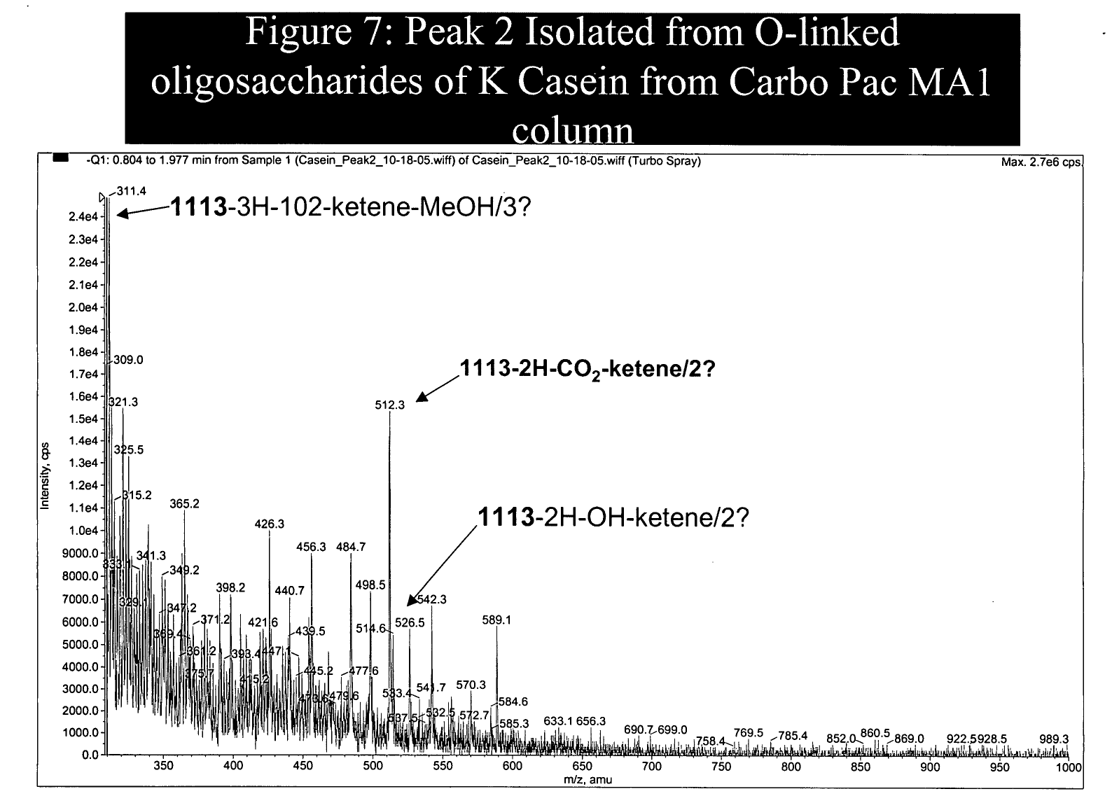

The cleaved off O-linked oligosaccharide alditols of Example 3 were further separated on a Carbo Pac MA1 ion exchange column. Two separate fractions were collected. FIGS. 6 and 7 depict ESI-mass spectra of each fraction in negative ion mode.

FIG. 6 shows the mass of the non-degraded NeuAc-NeuAc-Glc-HexNAc (SEQ ID NO:1) O-linked oligosaccharide and degradation products thereof. The mass spectrum of the fraction appears similar to the mass spectrum of the entire sample shown in FIG. 5. The second fraction depicted in FIG. 6, however, reveals that an additional cleaved-off O-linked oligosaccharide is present in the sample. Based on the mass spectrum, the oligosaccharide may be NeuAc-NeuAc-Fuc-Glc-HexNAcol (SEQ ID NO:2).

What is claimed is:

1. A method of detecting N-linked and O-linked oligosaccharides from a glycoprotein including N-linked and O-linked oligosaccharides, the method comprising:
enzymatically cleaving one or more N-linked oligosaccharides but not the O-linked oligosaccharides from the glycoprotein to form cleaved-off N-linked oligosaccharides and residual glycoprotein;
immobilizing the residual glycoprotein on a solid substrate;
separating the cleaved-off N-linked oligosaccharides from the residual glycoprotein;
cleaving O-linked oligosaccharides from the separated residual glycoprotein to form cleaved-off O-linked oligosaccharides and a residual protein;
separating the cleaved-off O-linked oligosaccharides from the residual protein; and
detecting said separated N-linked and O-linked oligosaccharides.

2. The method of claim 1, wherein the enzymatically cleaving step comprises contacting the glycoprotein with an enzyme selected from the group consisting of PNGase F, PNGase A and mixtures thereof.

3. The method of claim 1, wherein said step of separating the cleaved-off N-linked oligosaccharides is performed by flowing an aqueous liquid stream past said solid substrate to carry away said cleaved-off N-linked oligosaccharides from said residual glycoprotein.

4. The method of claim 3, wherein the solid substrate is disposed in a housing.

5. The method of claim 4, wherein the housing comprises a column.

6. The method of claim 5, wherein the solid substrate is a cation exchange medium.

7. The method of claim 6, wherein the cation exchange medium is in ammonium form.

8. The method of claim 1, wherein the steps are performed in an aqueous liquid.

9. The method of claim 1, further comprising reducing the cleaved-off N-linked oligosaccharides to form cleaved-off N-linked oligosaccharide alditols.

10. The method of claim 9, wherein the reducing step comprises contacting the cleaved-off N-linked oligosaccharide with a borohydride.

11. The method of claim 10, wherein the borohydride is NaBH₄ and the borohydride is converted to NaH₂BO₃.

12. The method of claim 11, further comprising replacing the sodium in said NaH₂BO₃ with NH₄⁺ ions to form NH₄H₂BO₃.

13. The method of claim 1 in which said cleaved-off N-linked oligosaccharides comprise at least a first and a second cleaved-off N-linked oligosaccharide, said method further comprising separating said first and second cleaved-off N-linked oligosaccharides.

14. The method of claim 13, wherein the step of separating said first and second N-linked oligosaccharides is performed by chromatography.

15. The method of claim 1, wherein the step of separating the cleaved-off O-linked oligosaccharides comprises immobilizing the residual glycoprotein on a second solid substrate.

16. The method of claim 1, wherein said detecting step comprises determining the monosaccharide composition of at least one of said cleaved-off N-linked oligosaccharides.

17. The method of claim 1, further comprising determining the sequence of at least one of the cleaved-off N-linked oligosaccharides.

18. The method of claim 1, wherein detecting step is performed by mass spectrometry or HPLC.

19. The method of claim 1, wherein the step of cleaving O-linked oligosaccharides from the residual glycoprotein comprises reductive beta-elimination of the O-linked oligosaccharide from the residual glycoprotein.

20. The method of claim 19, wherein the step of cleaving the O-linked oligosaccharides from the residual glycoprotein is performed at a pH between about 9.1 and 9.8 or about 10.2 and 11.4.

21. The method of claim 20, wherein the step of cleaving the O-linked oligosaccharides from the residual glycoprotein is performed in an NH₄OH/NaBH₄ solution.

22. The method of claim 1, wherein said step of separating the cleaved-off O-linked oligosaccharides from the residual glycoprotein is performed by flowing a liquid stream past said solid substrate after cleaving the O-linked oligosaccharides from the residual protein to carry away said O-linked oligosaccharides from the residual protein.

23. The method of claim 1, further comprising reducing the cleaved-off O-linked oligosaccharides.

24. The method of claim 1 in which said cleaved-off O-linked oligosaccharides comprise at least a first and a second cleaved-off O-linked oligosaccharide, said method further comprising separating said first and second O-linked oligosaccharides by chromatography.

25. The method of claim 24, wherein said step of separating said first and second O-linked oligosaccharides is performed on a column.

26. The method of claim 1, further comprising detecting at least one of said cleaved-off O-linked oligosaccharides.

27. The method of claim 1, wherein said detecting step is performed by mass spectrometry or HPLC.

28. A kit for separating N-linked oligosaccharides and O-linked oligosaccharides from a glycoprotein containing the N-linked and O-linked oligosaccharides, the kit comprising:
an enzyme capable of cleaving N-linked oligosaccharides but not O-linked oligosaccharides from said glycoprotein; and
a solid substrate capable of immobilizing glycoproteins.


SEQUENCE LISTING (2 sequences)

SEQ ID NO: 1 - protein, 4 aa, Artificial
  Synthetic
  MOD_RES (1)..(2): The Xaa at positions 1 to 2 are N- acetylneuraminic acid
  MOD_RES (3)..(3): The Xaa at position 3 is glucose
  MOD_RES (4)..(4): The Xaa at position 4 is N-acetylhexosamine
XXXX

SEQ ID NO: 2 - protein, 5 aa, Artificial
  Synthetic
  MOD_RES (1)..(2): The Xaa at positions 1 to 2 are N- acetylneuraminic acid
  MOD_RES (3)..(3): The Xaa at position 3 is fucose
  MOD_RES (4)..(4): The Xaa at position 4 is glucose
  MOD_RES (5)..(5): The Xaa at position 5 is N-acetylhexosaminitol
XXXXX